(19) United States
(12) Patent Application Publication
(10) Pub. No.: US 2021/0290700 A1
(43) Pub. Date: Sep. 23, 2021

(54) ORALLY ADMINISTERED SUPPLEMENTS AND METHODS OF REDUCING ABSORPTION OF CALORIES AND MODIFYING THE NUTRITIONAL VALUES OF CONSUMED NUTRIENTS

(71) Applicant: N.M.B. MEDICAL APPLICATIONS LTD, Herzliya (IL)

(72) Inventors: Mordechay BEYAR, Tel Aviv (IL); Oren GLOBERMAN, Kfar-Shmaryahu (IL); Danielle VALES, Tel Aviv (IL)

(73) Assignee: N.M.B. MEDICAL APPLICATIONS LTD, Herzliya (IL)

(21) Appl. No.: 17/264,821

(22) PCT Filed: Aug. 1, 2019
(86) PCT No.: PCT/IL2019/050869

§ 371 Date: Jan. 31, 2021

Related U.S. Application Data
(60) Provisional application No. 62/712,988, filed on Aug. 1, 2018.

(51) Int. Cl. A61K 35/747; A61K 9/00

(52) U.S. Cl. CPC A61K 35/747; A61K 9/0053

(57) ABSTRACT

Methods and orally administered supplements are provided, which use bacterial strains to reduce absorption of calories by a subject from consumed nutrients. The bacterial strain(s) are selected to metabolize at least part of the nutrient(s) consumed by the subject, and may be selected to be excreted and/or to have metabolic products that are excreted by the subject—making some of the consumed calories unavailable to the subject and/or reducing any of a glycemic, a cholesterol/lipids level and a protein value of the consumed nutrient(s). Appropriate coatings or covers may be applied to protect the bacteria from gastric acids, and the specific strains may be adjusted to increase their nutrient metabolism, improve their resistance to intestinal chemical conditions and optimize bacterial communities that may have synergic effects in nutrient metabolism, in a way that makes some of the calories unavailable to the subject.

### BACKGROUND OF THE INVENTION

### 1. Technical Field

The present invention relates to the field of weight reduction, and more particularly, to reduction of absorption of calories and/or modifying the nutritional values of consumed nutrients.

### 2. Discussion of Related Art

Overweight is one of the most common modern society health challenges, resulting from over-eating and the lack of physical activity in the modern lifestyle habits. A wide range of diets and medical treatments have been suggested, but no long-term weight reduction is easily achievable. Various prebiotic strains have been shown to have health benefits, possibly through their complex and poorly understood effects and interactions with the subject's intestinal flora (microbiome) and/or through modifications of bodily satiety/metabolic signals related, e.g., to absorption and/or storage of fat. In addition, certain diseases are related to specific nutrients consumption (e.g., diabetes to sugar, vascular diseases to cholesterol, renal diseases to proteins). The absorption of consumed nutrients is also related to the gut flora.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a method comprising administering, orally, at least one strain of bacteria that is selected to metabolize at least part of at least one nutrient consumed by a subject, and reducing absorption of calories by the subject and/or the nutrient value absorbed by the subject due to the consumption, through the bacterial metabolism of the at least one nutrient. For example, various embodiments reduce the glycemic index and/or the glycemic peak value for a diabetic subject with hyperglycemia and/or reduce cholesterol for a subject with hyperlipidemia and/or reduce the protein and/or amino acid value for a subject with renal disease due to the consumption, through the bacterial metabolism of the at least one nutrient.

One aspect of the present invention provides an orally administered supplement comprising at least one strain of bacteria that is selected to metabolize at least part of at least one nutrient consumed by a subject, wherein the orally administered supplement is configured to reduce absorption of calories by the subject and/or a nutrient value absorbed due to the consumption, through the bacterial metabolism of the at least one nutrient and/or excretion of the bacterial and their byproducts.

In certain embodiments, the at least one strain of bacteria may be selected to be excreted and/or to have metabolic products that are excreted by the subject.

One aspect of the present invention provides a method comprising administering, orally, at least one strain of bacteria that is selected to grow by increase in biomass and/or cell division by utilizing at least one nutrient consumed by a subject, wherein the biomass and/or byproducts of the bacterial growth are excreted by the subject to reduce the absorption of available calories and/or reduce the glycemic index and/or cholesterol/lipids level and/or protein value of the consumed nutrients.

One aspect of the present invention provides a method comprising administering, orally, at least one strain of bacteria that is selected to convert at least a part of one nutrient consumed by a subject to indigestible matter, wherein the at least one converted part of a nutrient is excreted by the subject to reduce the absorption of available calories and/or reduce glycemic index and/or cholesterol/lipids level and/or protein value of the consumed nutrients.

One aspect of the present invention provides a method comprising administering, orally, at least one strain of bacteria that is selected to convert at least one first nutrient consumed by a subject to at least one different second nutrient, to reduce the availability of the at least one first nutrient to the subject.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1 is a high-level schematic block diagram of the administration and operation in a subject's digestive system of an orally administered supplement, according to some embodiments of the invention.

FIG. 2 is a high-level flowchart illustrating a method, according to some embodiments of the invention.

FIGS. 3A-3D provide an experimental comparison of four Lactobacillus species with respect to their growth ratios in glucose-containing cultures.

FIGS. 4A-4D provide an experimental comparison of four Lactobacillus species with respect to their growth ratios in cultures containing sucrose, lactose, canola oil and olive oil.

FIGS. 5A-5D provide an experimental comparison of two Lactobacillus species with respect to their growth ratios in cultures containing glucose and sucrose at different concentrations, ranging between 0 and 2%.

FIGS. 6A-6D provide an experimental comparison of four Lactobacillus species with respect to their growth ratios in cultures containing oleic acid at different concentrations, of 0, 0.1% and 0.2%.

FIGS. 7A-7F provide an experimental comparison of four Lactobacillus species and combination thereof with respect to their growth ratios in cultures containing 1% glucose and oleic acid.

FIGS. 8A-8C provide an experimental comparison of three Lactobacillus species with respect to their growth ratios in cultures containing bile salts and/or oleic acids and/or emulsifier(s).

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Methods and orally administered supplements are provided, which use bacterial strains to reduce absorption of calories and/or reduce glycemic index and/or cholesterol/lipids level and/or protein value by a subject from consumed nutrients. The bacterial strain(s) are selected to metabolize at least part of the nutrient(s) consumed by the subject, and may be selected to be excreted and/or to have metabolic products that are excreted by the subject—making some of the consumed calories and/or nutrient(s) unavailable to the subject. Appropriate coatings or covers may be applied to protect the bacteria from gastric acids and/or other adverse conditions or compounds (e.g., alkaline conditions in the duodenum), and the specific strains may be adjusted to increase their nutrient metabolism, improve their resistance to intestinal chemical conditions and optimize bacterial communities that may have synergic effects in nutrient metabolism, in a way that makes some of the calories and/or nutrient(s) unavailable to the subject. In various embodiments, the protective cover may be provided by macro-encapsulation, micro-encapsulation and/or a combination of both. For example, macro encapsulation may provide protection during the passing through gastric acidity and micro encapsulation may provide protection during the pass through the alkaline conditions in the duodenum. It is noted that the encapsulation may be applied to a single strain of bacteria or to a group of bacteria strains. In various embodiments, the protective cover may be configured to provide a specified duration for the encapsulation to dissolve (and as a result decompose throughout the digestive system) and release the bacteria. The specified duration may be different for the macro- and micro-encapsulations. For example, any of the encapsulations may dissolve in any of the duodenum, the small intestine and/or the colon.

Various embodiments provide methods for weight reduction by using organisms (e.g., bacteria) that when present in the subject's intestine can reduce a part of the subject's alimentary energy intake and caloric absorption, e.g., using fast growing organisms that utilize energy taken from the subject's food for their own growth, increasing the organisms' biomass which is later excreted from the subject's body as feces and/or using organisms that digest the subject's food and convert it to indigestible matter which is later excreted from the subject's body (with or without the organisms themselves). Any of the embodiments may prevent a certain amount of the subject's energy intake to be absorbed, resulting in reduction of caloric availability and weight reduction of the subject. Clearly, the organisms used are selected to be safe for consumption.

Various embodiments provide methods for weight reduction that comprise administering, orally, at least one strain of bacteria that is selected to grow by increase in biomass and/or cell division by utilizing at least one nutrient consumed by a subject, wherein the biomass and/or byproducts of the bacterial growth are excreted by the subject to reduce the absorption of available calories and/or reduce glycemic index and/or cholesterol/lipids level and/or protein value of the consumed nutrients and food.

Various embodiments provide methods for weight reduction that comprise administering, orally, at least one strain of bacteria that is selected to convert at least one nutrient consumed by a subject to indigestible matter, wherein the at least one converted nutrient is excreted by the subject to reduce the absorption of available calories by the subject and/or reduce the glycemic index and/or cholesterol/lipids level and/or protein value of the consumed nutrients and food.

Various embodiments provide methods for weight reduction that comprise administering, orally, at least one strain of bacteria that is selected to convert at least one first nutrient consumed by a subject to at least one different second nutrient, to reduce the availability of the at least one first nutrient to the subject.

Disclosed embodiments may comprise one or more of the following effects, namely bacterial growth (by biomass and/or cell division), conversion of at least one nutrient to indigestible matter (e.g., conversion of glucose to cellulose) and/or conversion of at least one nutrient to another nutrient (e.g., conversion of glucose to amino acids or lipids). Corresponding caloric may be achieved by the bacterial use of the nutrients as energy for existence and reproduction and/or by the bacterial conversion of nutrients into indigestible matter such as bacterial byproducts. Either or both bacterial mass and the indigestible material may be excreted and thus removed from the body with their associated caloric and/or nutritional value. Additionally, in various embodiments, the administered bacteria may modify the availability of certain nutrients to the body, to regulate physiological reactions other than absorption of calories, e.g., sugar and/or lipid levels in the blood and/or protein value available for subject's absorption.

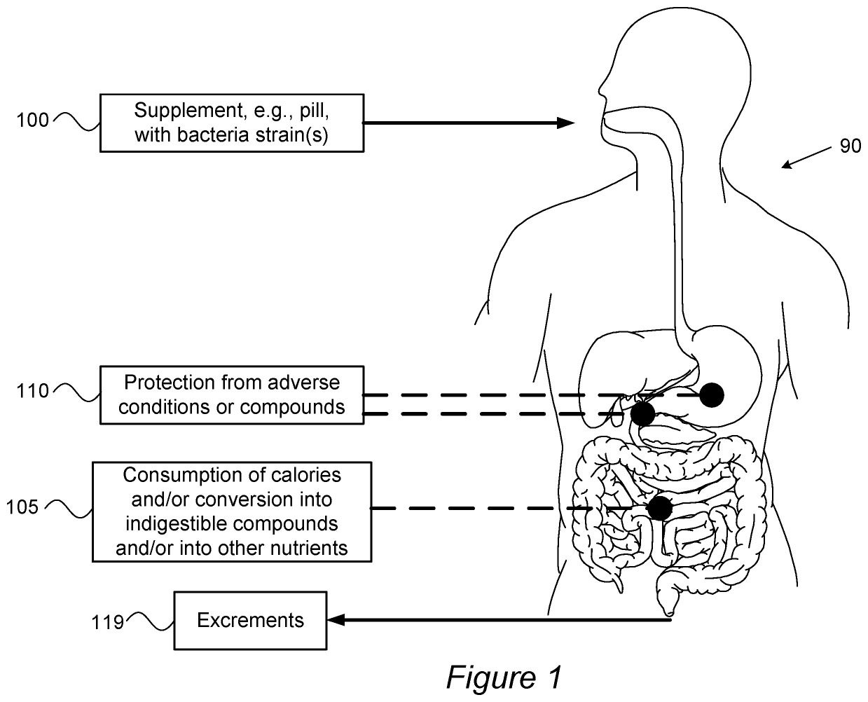

FIG. 1 is a high-level schematic block diagram of the administration and operation in a subject's digestive system of an orally administered supplement 100, according to some embodiments of the invention. Supplement 100, such as a pill or any other administrable form of at least one strain of bacteria, may be orally administered to subject 90 (e.g., a person or an animal), possibly within a cover (e.g., a pill's protective coating) that provides protection 110 from gastric acid and/or other adverse conditions or compounds (e.g., alkaline conditions in the duodenum) in the stomach and enables supplement 100 to reach, at least partly active, the subject's small intestine (and/or possible the subject's large intestine). The bacterial strain(s) is selected to metabolize 105 at least part of at least one nutrient consumed by subject 90, and may be further selected to be excreted 119 and/or to have metabolic products that are excreted 119 by subject 90, e.g., as feces. Orally administered supplement 100 is configured to reduce absorption of calories by subject 90 and/or reduce glycemic index and/or cholesterol/lipids level and/or protein value of the consumed food, through the bacterial metabolism of the nutrient(s). It is noted that the glycemic index provides a relative ranking of carbohydrates in food that relates to their effect on a subject's blood glucose levels.

For example, the bacterial strain(s) is selected to metabolize sugar (e.g., Lactobacillus plantarum or possibly other Lactobacillus spp. or combinations thereof) to reduce the absorption of calories of subject 90 and/or possibly to reduce the subject's blood sugar level. Alternatively or complementarily, bacterial strain(s) may be selected to metabolize lipids, to reduce the absorption of calories of subject 90 and/or possibly to reduce the subject's blood cholesterol and/or blood lipid level. Alternatively or complementarily, bacterial strain(s) may be selected to metabolize proteins. In certain embodiments, the bacterial strain(s) may be selected to convert one or more nutrient into one or more other nutrient, e.g., convert carbohydrates into lipids to reduce blood sugar level and/or possibly to reduce the subject's protein value available for subject absorption.

In various embodiments, the bacterial strain(s) may be adjusted, prior to the oral administration, to enhance its metabolism of nutrient(s), e.g. in vitro, in presence of nutrient(s), e.g., by applying a classical genetic process.

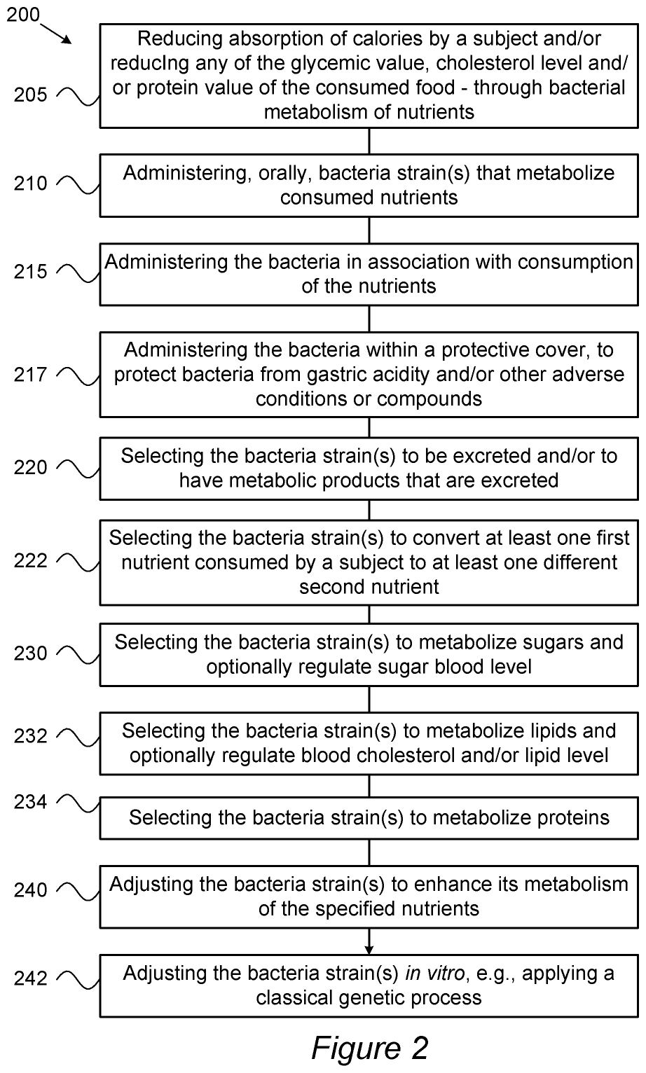

FIG. 2 is a high-level flowchart illustrating a method 200, according to some embodiments of the invention. The method stages may be carried out with respect to orally administered supplement 100 described above, which may optionally be configured to implement method 200. Method 200 may comprise the following stages, irrespective of their order.

Method 200 comprises administering, orally, at least one strain of bacteria that is selected to metabolize at least part of at least one nutrient consumed by a subject (stage 210), wherein the at least one strain of bacteria may be further selected to be excreted and/or to have metabolic products that are excreted by the subject (stage 220), possibly converting at least one first nutrient consumed by a subject to at least one different second nutrient that may be excreted (stage 222); and reducing absorption of calories by the subject and/or reduce the glycemic index and/or cholesterol/lipids level and/or protein value of the consumed food due to the consumption, through the bacterial metabolism of the at least one nutrient (stage 205). In certain embodiments, the bacteria may convert one or more nutrient to one or more other nutrient, e.g., convert carbohydrates to lipids to reduce blood sugar level (or possibly caloric intake).

In certain embodiments, method 200 may comprise carrying out oral administration 210 in association with consumption of the at least one nutrient by the subject (stage 215), e.g., simultaneously, shortly before and/or shortly after the consumption, or possibly irrespective of the time of nutrient consumption. In certain embodiments, administering 210 may be carried out within a protective cover (e.g., a pill's protective coating), configured to protect the at least one strain of bacteria from gastric acidity (stage 217) and/or other adverse conditions or compounds (e.g., alkaline conditions in the duodenum).

In various embodiments, the protective cover may be provided by macro-encapsulation, micro-encapsulation and/or a combination of both. For example, macro encapsulation may provide protection during the passing through gastric acidity and micro encapsulation may provide protection during the pass through the alkaline conditions in the duodenum. It is noted that the encapsulation may be applied to a single strain of bacteria or to a group of bacteria strains. In various embodiments, the protective cover may be configured to provide a specified duration for the encapsulation to dissolve (and as a result decompose throughout the digestive system) and release the bacteria. The specified duration may be different for the macro- and micro-encapsulations. For example, any of the encapsulations may dissolve in any of the duodenum, the small intestine and/or the colon.

In certain embodiments, method 200 may comprise selecting the at least one strain of bacteria to be excreted in the subject's feces. Bacterial digestion of nutrient(s) from the subject's food may increase the bacteria biomass, preventing or reducing absorption of calories from the nutrient(s) by the subject.

In certain embodiments, method 200 may comprise selecting the at least one strain of bacteria to metabolize the at least part of the at least one nutrient into metabolic products that are excreted in the subject's feces. Bacterial conversion of nutrient(s) from the subject's food may yield indigestible matter or compounds, preventing or reducing absorption of calories from the nutrient(s) by the subject and/or reducing the glycemic index and/or cholesterol/lipids level and/or protein value of the consumed food.

In certain embodiments, the at least one nutrient comprises sugar (e.g., glucose) and the at least one strain of bacteria comprises Lactobacillus plantarum, selected to metabolize sugars, and method 200 may further comprise reducing a blood sugar level in the subject by the sugar consumption of L. plantarum (stage 230).

In certain embodiments, the at least one nutrient comprises lipids (e.g., oleic acid) and the at least one strain of bacteria comprises L. casei selected to metabolize lipids, and method 200 may further comprise reducing a blood cholesterol and/or a blood lipid level in the subject by the lipid consumption of the bacteria (e.g., L. casei) (stage 232).

In certain embodiments, the at least one nutrient comprises proteins and the at least one strain of bacteria comprises corresponding strains, selected to metabolize proteins, and method 200 may further comprise reduction of absorption of calories in the subject and/or reduction of the amount of protein available for subject digestion by the protein consumption and/or conversion of proteins into other nutrients (stage 234).

In various embodiments, method 200 may further comprise adjusting the at least one strain of bacteria, prior to the administering, to enhance its metabolism of the at least one nutrient (stage 240). In certain embodiments, adjusting 240 may be carried out in vitro, in presence of the at least one nutrient, possibly by applying a classical genetic process (stage 242), e.g., repeatedly selecting the best strains with respect to certain conditions such as their consumption of sugars and/or lipids and their resistance to conditions in the digestive tract (e.g., gastric acidity, duodenal alkalinity). For example, at specified periods, the culture may be sorted under a microscope selecting about 10% biggest cells in the culture and/or dilution of the culture, which may then be subjected to further growth under the same conditions. For example, the culture may be diluted by a factor between 10-100 once a week for four weeks.

In certain embodiments, the used organisms (e.g., bacteria) may be selected and/or grown outside the body, e.g., lactic acid bacteria may be grown on a medium that mimics the small intestine (e.g., cultured at 37° C. for 6 hours). In various embodiments, the culture temperature may be between 25° C. to 42° C., e.g., between 35° C. to 38° C. The proliferative pH may be between 3.0 to 12.5, possibly about the pH of the host's small intestine. It is noted that a value modified by the term “about” is understood to encompass ±10% of the value. Oral administration may include protection of the bacteria supplement, e.g., using a gastric acid resistant coating that enables disintegration and spread of the bacterial strain(s) in the subject's small intestine. The bacteria may be selected to reproduce in the small intestine, e.g., flourishing within a few hours (e.g., between 6-8 hours) or possibly even faster (e.g., tens of minutes or seconds), preceding the absorption of nutrients by the subject's body. In case of delayed reproduction, the administered bacteria may still reduce the subject's absorption of calories and/or reduce the glycemic index and/or cholesterol/lipids level and/or protein value of the consumed food, with a certain delay.

In certain embodiments, oral administration may be carried out in devices such as coated pills, e.g., as food supplements or pharmaceuticals, as well as in the form of food additives and supplements, e.g., in various food products, e.g., dairy products or functional food. The supplements may comprise one or more bacterial strains, and may include additives such as lactic acid bacteria, probiotic, prebiotic, fibers or any other acceptable carrier. The oral administration may comprise one-time administration or be periodic, possibly over a prolonged period. The administration dosage may vary according to host preferences and/or effects such as preferred frequency of use and preferred reduction of absorption of calories by the subject and/or the reduction of the glycemic index and/or cholesterol/lipids level and/or protein value of the consumed food. In various embodiments, a one-day dosage may be divided into several sub-units so that may be administered per meal. In other cases, the dosage may be administered once every several days. Additionally or alternatively, the bacterial strain(s) may be selected to self-reproduce in the subject, and proliferate upon nutrients intake, requiring mere maintenance of a minimal population in the subject. The protective coating may comprise any enteric coating, which may be applied in various manners on a single strain of bacteria and/or on a group of several strains of bacteria. The cover may decompose after a predefined time according to the place in the digestive tract in which the metabolism takes place and/or after passing specified adverse conditions or compounds in the digestive tract, e.g., by spraying a coating solution onto a core, and may have specified resistance and disintegration durations, e.g., the enteric coating may be selected to have resistances, e.g., between (i) withstanding up to at least two hours in an artificial gastric juice such as HCl solution (pH 1) at 36-38° C., and (ii) decomposing within 30 minutes in an artificial intestinal juice such as KH₂PO₄ buffer solution (pH 6.8). In various embodiments, the reduced absorption of calories and/or the reduced glycemic index and/or cholesterol/lipids level and/or protein value may be about 5%, 10% or more that is turned into excreted bacterial mass and/or converted indigestible matter and/or a matter different than the originally administrated nutrient and may yield over prolonged use about 5%, 10% or higher body weight reduction. In certain embodiments, the reduced absorption of calories and/or glycemic index and/or cholesterol/lipids level and/or protein value may yield body weight and/or available nutrient reduction following short-term use.

The following experimental results were derived using the following bacterial strains: Lactobacillus bervis, L. fermentum, L. plantarum and L. casei. An additional strain L. acidophilus was found to be inferior in performance to these four strains and the results below do not include it. The cultures were grown on De Man, Rogosa and Sharpe (MRS) agar with different sugars and lipids added thereto at different concentrations, as specified below. In certain experiments, modified MRS agar—lacking dextrose—was used and indicated as “MRS W/O”. Bacterial growth was measured in terms of optical density (OD) at 20 minutes time steps.

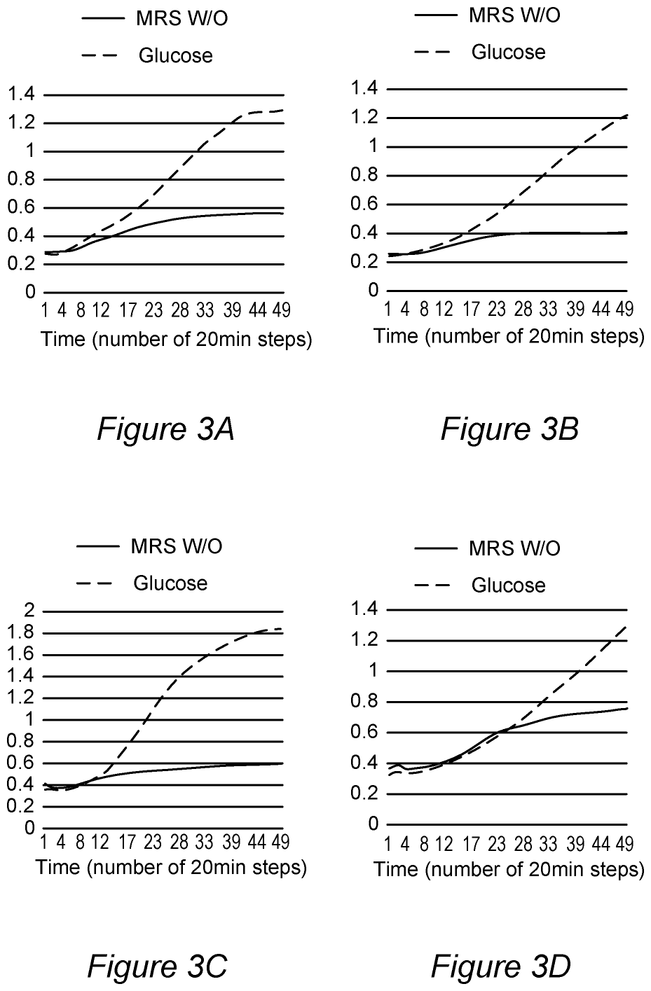

FIGS. 3A-3D provide an experimental comparison of four Lactobacillus species with respect to their growth ratios in glucose-containing cultures versus control cultures with no additives. Of the four species L. brevis, L. fermentum, L. plantarum and L. casei depicted in FIGS. 3A-3D, respectively, L. plantarum is seen to have the highest growth rates.

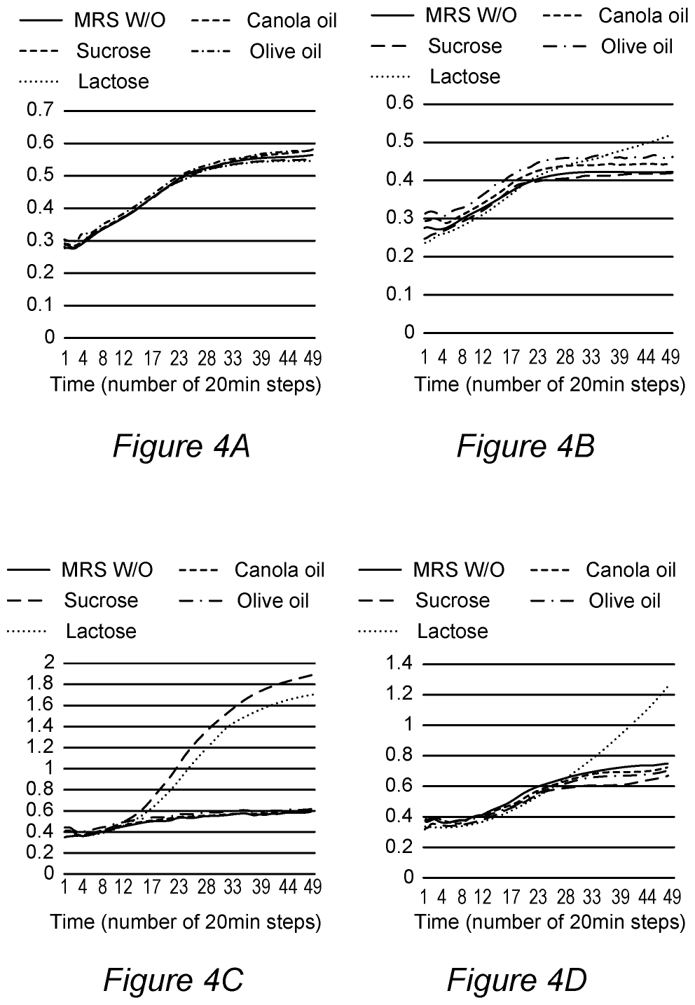

FIGS. 4A-4D provide an experimental comparison of four Lactobacillus species with respect to their growth ratios in cultures containing sucrose, lactose, canola oil and olive oil versus control cultures with no additives. Of the four species L. brevis, L. fermentum, L. plantarum and L. casei depicted in FIGS. 4A-4D, respectively, L. plantarum is seen to have the highest growth rates in the presence of sucrose and of lactose.

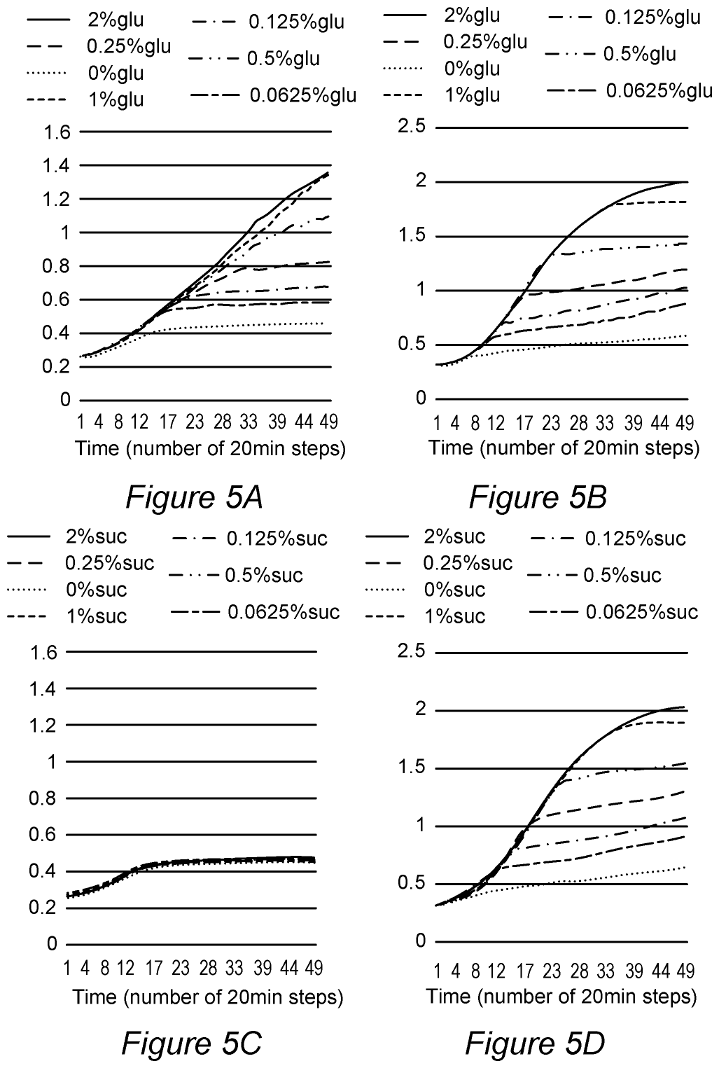

FIGS. 5A-5D provide an experimental comparison of two Lactobacillus species with respect to their growth ratios in cultures containing glucose (“glu”) and sucrose (“suc”) at different concentrations, ranging between 0 and 2%. FIGS. 5A and 5B provide the growth of L. fermentum and L. plantarum, respectively, with glucose, and FIGS. 5C and 5D provide the growth of L. fermentum and L. plantarum, respectively, with sucrose. In the former, L. plantarum is seen to have higher growth rates and yield higher concentrations for all glucose concentrations, in the latter L. plantarum is seen to have much higher growth rates and yield much higher concentrations for all sucrose concentrations, while L. fermentum remains limited in its growth. It is further noted that in the presence of glucose, the growth rate of L. plantarum, calculated using the slope of the graphs (0.3 OD increases, representing population growth of ten million bacteria, correspond to 0.25% glucose concentration increases), indicates that 100 million (10⁸) bacteria consume 25 grams of glucose in vitro. Extrapolating these results to typical bacterial doses for oral administration via pills of 10¹⁰ bacteria, each dose would correspond to the bacteria metabolizing 2.5 kg of glucose, reducing significantly the subject's absorption of calories. It is noted that these data provide a rough estimation in vitro, and that the actual sugar consumption in the body may be lower and may vary depending on various factors.

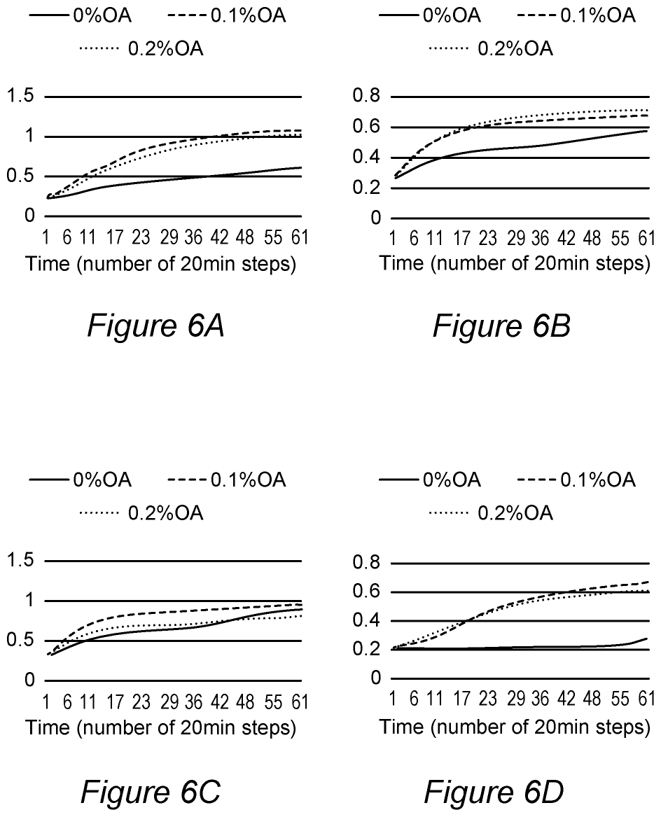

FIGS. 6A-6D provide an experimental comparison of four Lactobacillus species with respect to their growth ratios in cultures containing oleic acid (“OA”) at different concentrations, of 0, 0.1% and 0.2%. FIGS. 6A, 6B, 6C and 6D provide the growth of L. brevis, L. fermentum, L. plantarum and L. casei, respectively, and illustrate that L. casei yields that largest increase in growth in the presence of oleic acid.

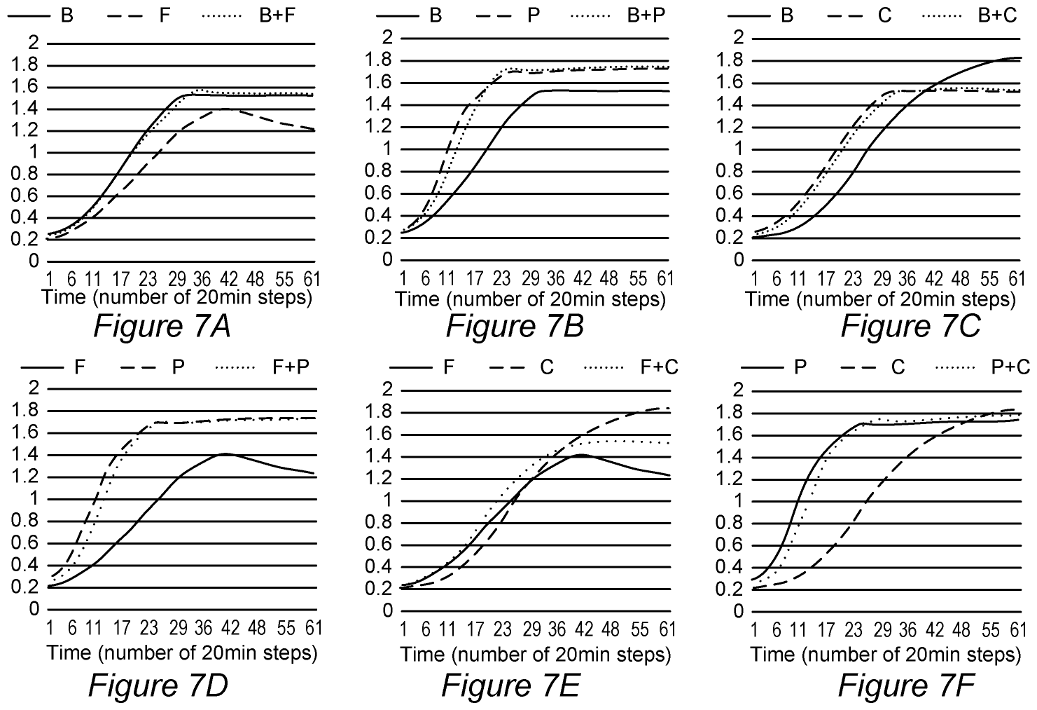

FIGS. 7A-7F provide an experimental comparison of four Lactobacillus species and combination thereof with respect to their growth ratios in cultures containing 1% glucose and oleic acid. FIGS. 7A-7F provide growth rates of L. brevis (“B”), L. fermentum (“F”), L. plantarum (“P”) and L. casei (“C”), and their pair-wise combinations. The cultures of L. casei provide the highest growth rates.

The oleic acid experiments resulted in showing that L. brevis, L. fermentum and L. plantarum can grow and utilize oleic acid, with L. fermentum requiring additional glucose and L. brevis and L. plantarum being able to metabolize oleic acids in the absence of glucose as well. Moreover, the graphs indicate that the different Lactobacillus species do not reduce each other's growth. The mixing experiments did not show any specific advantageous combination of the tested bacteria.

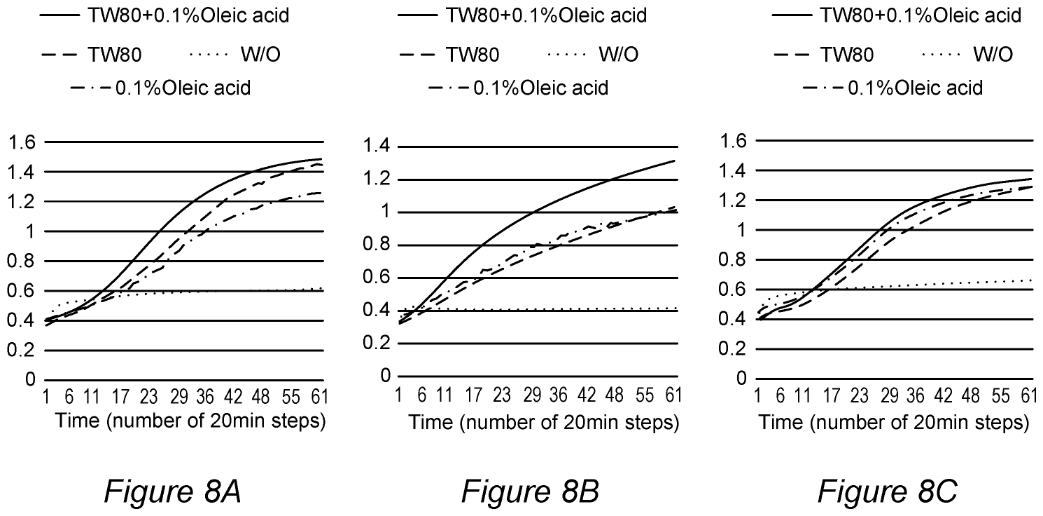

FIGS. 8A-8C provide an experimental comparison of three Lactobacillus species with respect to their growth ratios in cultures containing bile salts (0.1%) and/or oleic acids and/or emulsifier(s). The used emulsifier was Tween 80 (polysorbate 80, polyoxyethylene sorbitan monooleate) and is indicated as “TW80”. FIGS. 8A, 8B and 8C provide the growth of L. brevis, L. fermentum and L. plantarum, respectively, and illustrate that all species grow in the presence of bile salts. The bile salt experiments indicate that the tested bacteria strains can grow in presence of up to 0.1% bile salts. Moreover, the results show that oleic acid and/or the emulsifier support and enhance bacterial growth in presence of the bile salts.

In certain embodiments, the bacterial strains may be adjusted to grow in presence of glucose and sucrose is possible, applying a classic genetic process to select the best strains with respect to their consumption of sugars. For example, the population of L. plantarum was increased and stabilized at a high level of glucose and sucrose metabolism at 1.8OD after ca. 8 hours.

Advantageously, disclosed methods and supplements for reducing the absorption of calories may lead to direct metabolism of consumed calories into bacterial biomass and/or indigestible metabolism products which may be excreted from the subject, making a part of the subject's absorption of calories unavailable and/or reduce the glycemic index and/or cholesterol/lipids level and/or protein value of the consumed food. In contrast to former studies, the disclosed effects are direct and simple to understand and control, as they involve few if any interactions with the subject's physiological signaling system, and provide a direct and measurable path to reducing the absorption of calories. Additional advantages provided by disclosed embodiments include the ability to directly reduce the glycemic index for a diabetic subject with hyperglycemia and/or to directly reduce cholesterol for a subject with hyperlipidemia—using the bacterial metabolism and nutrient conversion to achieve the direct reduction. Advantageously, disclosed embodiments may be utilized to reduce and/or delay absorption of nutrients that relate to specific conditions, such as sugar (for handling diabetes), cholesterol (for handling vascular diseases) and/or proteins (for handling renal diseases).

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A method comprising:
administering, orally, at least one strain of bacteria that is selected to metabolize at least part of at least one nutrient consumed by a subject, and
reducing absorption of calories by the subject due to the consumption, and/or reducing a glycemic index and/or a cholesterol/lipids level and/or a protein value of the consumed at least one nutrient—through the bacterial metabolism of the at least one nutrient.

2. (canceled)

3. (canceled)

4. The method of claim 1, further comprising selecting the at least one strain of bacteria to be excreted in the subject's feces to reduce absorption of the calories from the at least one nutrient by the subject.

5. The method of claim 1, further comprising selecting the at least one strain of bacteria to metabolize the at least part of the at least one nutrient into metabolic products that are excreted in the subject's feces.

6. The method of claim 1, wherein the at least one nutrient comprises sugar and the at least one strain of bacteria comprises L. plantarum.

7. The method of claim 6, further comprising reducing a blood sugar level and/or blood sugar peak level in the subject by the sugar consumption of L. plantarum.

8. (canceled)

9. (canceled)

10. (canceled)

11. The method of claim 1, further comprising adjusting the at least one strain of bacteria, prior to the administering, to enhance its metabolism of the at least one nutrient.

12. The method of claim 11, wherein the adjusting is carried out in vitro, in presence of the at least one nutrient.

13. (canceled)

14. The method of claim 1, wherein the administering is carried out within a protective cover, configured to protect the at least one strain of bacteria from gastric acidity and/or duodenum alkalinity and/or other adverse conditions or compounds.

15. The method of claim 14, wherein the protective cover comprises macro-encapsulation and/or micro-encapsulation of the at least one strain of bacteria, wherein the macro-encapsulation and/or micro-encapsulation are each configured to protect the bacteria under specified conditions in the subject's digestive tract and are each further configured to have corresponding dissolution conditions and timing.

16. (canceled)

17. An orally administered supplement comprising:
at least one strain of bacteria that is selected to metabolize at least part of at least one nutrient consumed by a subject,
wherein the orally administered supplement is configured to reduce absorption of calories by the subject due to the consumption, through the bacterial metabolism of the at least one nutrient.

18. The orally administered supplement of claim 17, wherein the at least one strain of bacteria is selected to be excreted and/or to have metabolic products that are excreted in the subject's feces.

19. (canceled)

20. (canceled)

21. The orally administered supplement of claim 17, wherein the at least one nutrient comprises sugar and the at least one strain of bacteria comprises L. plantarum, wherein the orally administered supplement is configured to reduce a blood sugar level in the subject by the sugar consumption of L. plantarum.

22. (canceled)

23. (canceled)

24. The orally administered supplement of claim 17, wherein the at least one strain of bacteria is adjusted, prior to the oral administration, to enhance its metabolism of the at least one nutrient.

25. (canceled)

26. The orally administered supplement of claim 24, wherein the adjustment is carried out by applying a classical genetic process.

27. (canceled)

28. (canceled)

29. (canceled)

30. A method comprising administering, orally, at least one strain of bacteria that is selected to grow by increase in biomass and/or cell division by utilizing at least one nutrient consumed by a subject, wherein the biomass and/or byproducts of the bacterial growth are excreted by the subject to reduce absorption of available calories by the subject and/or reduce a glycemic index and/or a cholesterol/lipids level and/or a protein value of the consumed at least one nutrient.

31. (canceled)

32. (canceled)

33. (canceled)

34. (canceled)

35. The method of claim 30, further comprising selecting the at least one strain of bacteria to metabolize the at least part of the at least one nutrient into metabolic products that are excreted in the subject's feces.

36. The method of claim 30, wherein the at least one strain of bacteria comprises L. plantarum and/or L. casei.

37. The method of claim 30, further comprising adjusting the at least one strain of bacteria, prior to the administering, to enhance its metabolism of the at least one nutrient.

38. (canceled)

39. The method of claim 37, wherein the adjusting is carried out by applying a classical genetic process.

40. The method of claim 30, wherein the administering is carried out within a protective cover, configured to protect the at least one strain of bacteria from gastric acidity and/or duodenum alkalinity and/or other adverse conditions or compounds.

41. (canceled)

42. (canceled)
